(19) United States
(12) Patent Application Publication
(10) Pub. No.: US 2008/0293161 A1
(43) Pub. Date: Nov. 27, 2008

(54) Detection of Carbohydrate Biomarkers

(75) Inventors: Susan L. Deutscher, Columbia, MO (US); Thomas P. Quinn, Columbia, MO (US); Edward R. Sauter, Columbia, MO (US)

Correspondence Address: SENNIGER POWERS LLP, 100 NORTH BROADWAY, 17TH FLOOR, ST LOUIS, MO, 63102, US

(73) Assignee: The Curators of the University of Missouri, Columbia, MO (US)

(21) Appl. No.: 12/187,159

(22) Filed: Aug. 6, 2008

Related U.S. Application Data
(63) Continuation of application No. 11/416,117, filed on May 2, 2006.
(60) Provisional application No. 60/676,764, filed on May 2, 2005.

(51) Int. Cl. G01N 33/566; G01N 33/00

(52) U.S. Cl. 436/501; 436/63

(57) ABSTRACT

The present invention generally relates to detection of carbohydrate biomarkers in nipple aspirate fluid samples. One aspect of the invention is a method for assaying a nipple aspirate fluid for the presence of TF or Tn carbohydrate biomarker. The assay generally employs an immobilized capture agent specific for TF or Tn and can be further coupled to either direct or indirect detection of bound TF or Tn carbohydrate biomarker through the use of a labeled binding agent.

This application is a continuation of U.S. application Ser. No. 11/416,117, filed May 2, 2006, which claims the benefit of U.S. Provisional Application Ser. No. 60/676,764, filed May 2, 2005, the entire content of each of which is hereby incorporated herein by reference.

This invention was made with Government support under DAMD 17-03-1-0509 awarded by US Department of Defense, Breast Cancer Research Program. The Government has certain rights in the invention.

### FIELD OF THE INVENTION

The present invention generally relates to detection of carbohydrate biomarkers present in nipple aspirate fluid.

### BACKGROUND

Early detection of breast cancer facilitates successful disease treatment and management. Recently, nipple aspiration of breast fluid has shown promise in assisting in the detection of in situ and invasive breast cancer. Nipple aspirate fluid (NAF) can be obtained noninvasively and contains relatively high levels of proteins and lipids secreted from ductal and lobular epithelia, but only a small number of cancer cells in those patients with cancer (Glinsky (2001) Cancer Research 61: 4851-4857; Hsiung et al., Cancer Journal (2002) 8, 303-310). NAF may be obtainable from up to 95% of women (Sauter et al. (1997) Br Cancer 76: 494-501).

The Thomsen-Friedenreich (TF) and related Tn antigens, are found in breast carcinoma but not healthy breast tissue (Springer et al. (1980) Cancer 45: 2949-29541). In certain studies, greater than 85% of patients with ductal breast cancer were TF and Tn antigen positive while over 94% of patients with benign breast disease were negative (Springer et al. (1980) Cancer 45: 2949-29541; Springer (1984) Science 224: 1198-1206). TF [Galβ1→3GalNAc-] and Tn [3GalNAc-] are early differentiation carbohydrate antigens that are linked to Ser/Thr on glycoproteins and can be found on cancer-associated glycolipids and ceramides. Both TF and Tn antigen are covalently masked in healthy individuals but exposed and immunoreactive in greater than 90% of carcinoma patients. It has been reported that there is a positive correlation between the amount of TF and Tn antigens and the carcinoma's aggressiveness (Springer, (1984) Science 224: 1198-1206; Glinsky, (2001) Cancer Research 61: 4851-4857). It is also known that the TF antigen, present on the surface of breast carcinoma cells, plays an important role in the early stages of metastatic deposition (Glinsky (2001) Cancer Research 61: 4851-4857).

The TF antigen can be detected via the galactose oxidase-Schiff (GOS) reaction. The GOS reaction yields positive results in many malignancies, including carcinomas of the breast, as well as the lung, pancreas, ovary, thyroid, stomach, and colon. This reaction has been studied in breast tissue sections and has been reported to yield positive results in breast carcinoma tissue and negative results in normal breast tissue, using a spectrophotometric assay system (Shamsuddin (1995) Cancer Res 55: 149-152).

The Tn antigen has been reported to be quantified in ascitic and pleural effusion samples from patients via a double-determinant immunolectin-enzymatic method that uses a monoclonal antibody “catcher” and an isolectin tracer (Freire et al. (2003) Oncology Reports 10: 1577-1585).

### SUMMARY OF THE INVENTION

The present invention is generally directed to an assay allowing determination of carbohydrate biomarkers present in nipple aspirate fluid. These biomarkers occur on protein, carbohydrate, and lipid molecules present in nipple aspirate fluid and can serve as indicators of breast cancer. The methods described herein facilitate the non-invasive detection of breast cancer at a variety of stages, and serve as a predictive measure that can signal an increased chance of developing cancer.

One aspect of the invention is a method for assaying carbohydrate biomarkers. In this method, a nipple aspirate fluid (NAF) or NAF derivative sample is assayed for the presence of TF and/or Tn carbohydrate biomarkers. Such assay generally employs a capture agent attached to a carrier to bind TF and/or Tn from the NAF sample. The presence of the bound carbohydrate biomarker(s) may then be detected directly or indirectly through the use of a labeled binding agent.

Another aspect of the present invention is a kit for detection of carbohydrate biomarkers in nipple aspirate fluid (NAF) or NAF derivative. The kit generally contains a capture agent capable of binding specifically to TF or Tn, a labeled binding agent, and instructions for use of the kit in accordance with the methods disclosed herein.

Other objects and features will be in part apparent and in part pointed out hereinafter.

### BRIEF DESCRIPTION OF THE DRAWINGS

FIGS. 1A and 1B are bar graphs depicting the concentration of TF and Tn antigens in non-cancer (FIG. 1A) and cancer (FIG. 1B) NAF samples, as indirectly determined by a serial antigen capture enzyme-linked immunosorbent assay (ELISA). Immunoassays were performed on the samples using monoclonal antibodies to specifically capture the TF and Tn antigens in NAF, the process of which is depicted in FIG. 2 and more fully described in Example 2.

FIG. 2 is a schematic representation of an example of the capture ELISA with indirect detection of TF- and/or Tn-antigen in NAF samples, more fully described in Example 2.

FIG. 3 is a bar graph depicting the concentration of TF and Tn antigens in cancer and non-cancer NAF samples. The concentrations were directly determined via a serial antigen capture enzyme-linked immunosorbent assay (ELISA), which are depicted in FIG. 4 and described more fully in Example 3.

FIG. 4 is a schematic representation of an example of the capture ELISA with direct detection of TF- and/or Tn-antigen in NAF samples, more fully described in Example 3.

### DETAILED DESCRIPTION OF THE INVENTION

The present invention is generally directed to an assay allowing determination of carbohydrate biomarkers that occur on protein, carbohydrate, and lipid molecules present in nipple aspirate fluid. In several embodiments, the carbohydrate biomarkers are indicators of breast cancer. The methods described herein facilitate the non-invasive detection of breast cancer at a variety of stages, and serve as a predictive measure that can signal an increased chance of developing cancer.

Generally, detection of carbohydrate biomarkers is according to an assay of nipple aspirate fluid. The various embodiments of the capture assay described herein provide the sensitivity necessary to detect low levels of TF or Tn carbohydrate biomarker in nipple aspirate fluid. The assay can detect TF, Tn, or both TF and Tn. Additionally, the assay detects any molecule displaying the carbohydrate biomarker of interest, including proteins, carbohydrates, and lipids. The sensitivity of the assay allows detection of carbohydrates and conjugated lipids, traditionally undetectable due to low abundance and unsuitability for western, PCR, or immunoblots.

The capture assay employs a TF- or Tn-specific capture agent to isolate, from the nipple aspirate fluid sample, proteins, lipids, or carbohydrates displaying a TF or Tn carbohydrate biomarker upon their surface. In one embodiment, the NAF sample is combined with an immobilized capture agent that specifically binds to TF or Tn carbohydrate biomarker. The capture agent can be, for example, a TF- or Tn-specific antibody, lectin, peptide, bacteriophage, or other molecule that has specific affinity for TF- or Tn-antigen, respectively. Preferably, the capture agent is a TF- or Tn-specific antibody and the assay is a capture immunoassay. The TF or Tn carbohydrate biomarkers within the nipple aspirate fluid, if any, will usually bind only a portion of the available attached capture agent binding sites. Carbohydrate biomarker bound to the capture agent can then be detected.

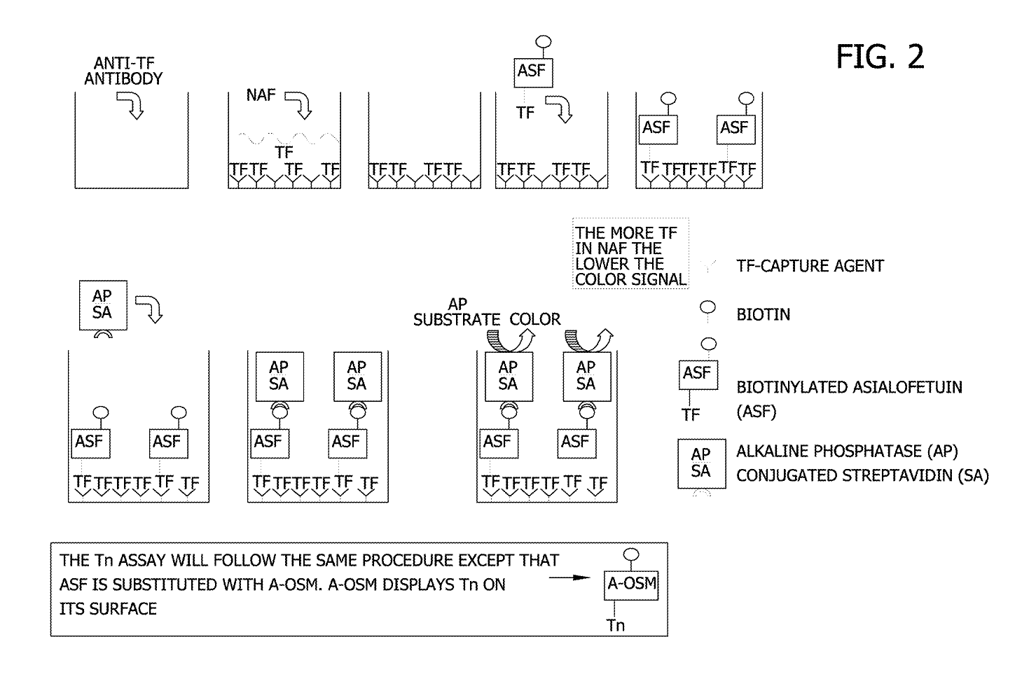

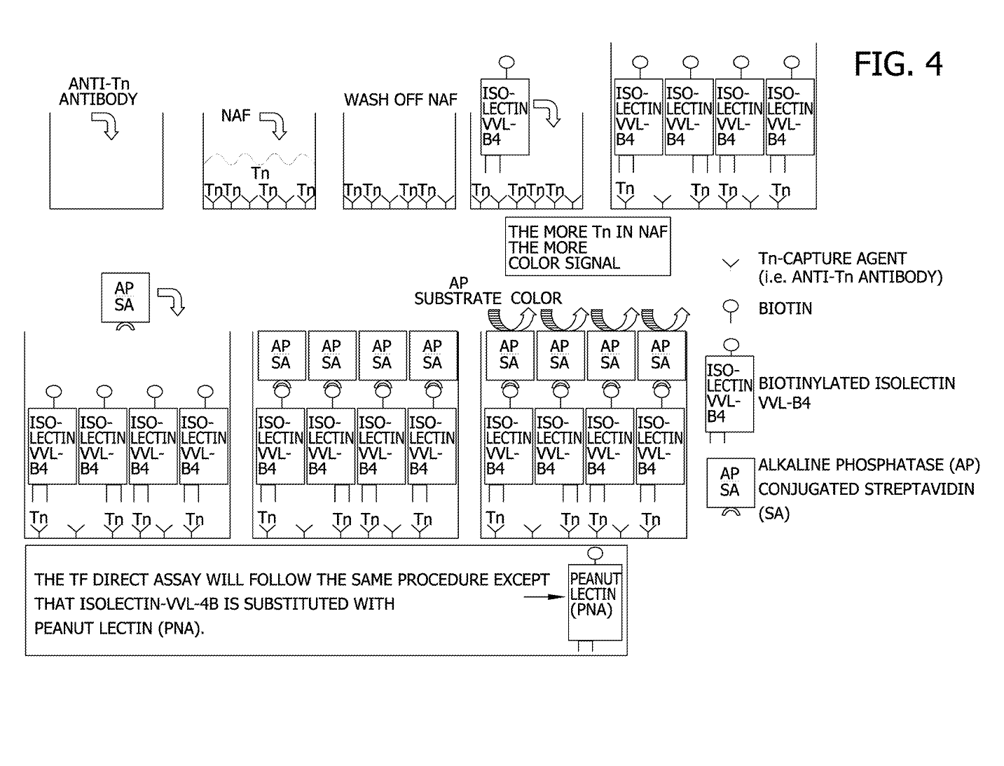

Carbohydrate biomarkers TF or Tn can be detected in the nipple aspirate fluid sample in a capture assay through indirect (see e.g. FIG. 2) or direct (see e.g. FIG. 4) detection means. For both indirect and direct detection, a binding agent, marked with an easily assayable tag, is brought into contact with the bound capture agent on the substrate. The tagged binding agent is generally provided in excess over its target. By comparing the assay signal (an indicator of tagged binding agent) to a standard curve of TF or Tn, one can quantify the amount of TF or Tn present in the sample of nipple aspirate fluid.

The difference between tagged binding agents for indirect and direct detection assays is the target for which the binding agent is specific. In indirect detection, the tagged binding agent binds to unoccupied capture agent binding sites. Quantitation of the amount of tag present correlates, inversely (i.e., indirectly), to the amount of TF or Tn carbohydrate biomarker captured from the nipple aspirate fluid sample (see FIG. 2). In direct detection, the tagged binding agent binds directly to TF or Tn, which in turn is bound to the immobilized capture agent. Quantitation of the amount of tag present correlates directly with the amount of TF or Tn carbohydrate biomarker captured from the nipple aspirate fluid sample (see FIG. 4).

The TF and Tn carbohydrate biomarker assay described herein can be employed to detect breast cancer at several different stages. These stages include, but are not limited to, ductal carcinoma in situ (DCIS); T1N1MO St-2A (where the tumor is less than 2 cm with lymph node involvement but no distant metastasis); T2N0/N1 MOSt-2A/B (where the tumor is greater than 2 cm with or without lymph node involvement but no distant metastasis); T3N1/N3 MOSt-3/3A (where the tumor is greater than 5 cm with lymph node involvement but no distant metastasis); and T4N1 MO St-3B (where the tumor is any size, with lymph node involvement but no distant metastasis). The TF and Tn carbohydrate biomarker assay described herein can also be employed to detect atypical hyperplasia (ADH), a condition considered as a risk factor for developing cancer where more abnormal cells are present. When used to detect atypical hyperplasia, the presence of TF and/or Tn carbohydrate biomarker would signal an increased chance of developing cancer.

Nipple Aspirate Fluid

Nipple aspirate fluid can be obtained through a variety of methods known in the art (see e.g., Sauter et al. (1997) Br J Cancer 76, 494-501). Nipple aspirate fluid bathes the ductal epithelial cells, which undergo malignant transformation in most forms of breast cancer. The aspirate contains exfoliated ductal epithelial cells, proteins, and lipids secreted from the ductal and lobular epithelia. Nipple aspirate fluid samples can be collected noninvasively, for example, by using a modified breast pump and/or by manual massage and expression (see e.g. Example 1). Various procedures can facilitate nipple aspirate fluid collection. These include warming the breast with, for example, a warm moist cloth or heating pad, and/or massage of the breast before, during, and/or after collection. Collection facilitated by massage can occur by manually expressing the breasts by placing hands flat around the base of the breast and squeezing down toward the tip of the nipple. During nipple aspirate fluid collection, keratin plugs can block aspiration. Dekeratinizing the nipple can be performed with rough gauze along with alcohol or Cerumenex 3%.

Nipple aspirate fluid may be diluted if, for example, the sample obtained is a small volume or the sample obtained is particularly viscous. Nipple aspirate fluid may also be diluted, for example, to standardize the sample obtained with other samples on a particular parameter. A nipple aspirate fluid sample may be diluted according to any of a number of known means, including, for example, the addition of an inert fluid, such as, for example, distilled water or a buffer such as PBS. Alternatively, nipple aspirate fluid may be concentrated by the removal of water if, for example, the volume of the sample is greater than desired for the assay. Water may be removed from a nipple aspirate fluid sample according to any of a number of known means, including, for example, use of a Savant SpeedVac®, lyophilization, or other means which do not remove, in addition to the water, a fraction of the nipple aspirate fluid which may contain the carbohydrate biomarker (e.g., the lipid fraction, protein fraction, carbohydrate fraction, or cellular debris).

Although generally less preferred, in one embodiment the nipple aspirate fluid may be fractionated to provide a nipple aspirate derivative which may then be analyzed for the carbohydrate biomarker. Fractionation of a nipple aspirate fluid sample may be accomplished by any of a number of known means, including, for example, centrifugation, ultrafiltration, chromatography, gel electrophoresis, and distillation. Thus, for example, a fraction may be obtained that, relative to nipple aspirate fluid as obtained from a patient (i.e., “complete” or “total” NAF), contains a ratio of lipid to protein, lipid to carbohydrate, or protein to carbohydrate that differs from the original sample.

Similarly, the nipple aspirate fluid may be concentrated, resulting in a concentrate that, relative to nipple aspirate fluid as obtained from a patient (i.e., “complete” or “total” NAF), contains a ratio of lipid to protein, lipid to carbohydrate, or protein to carbohydrate that differs from the original sample. In addition to the alteration of one or more of these ratios, the concentrate may also contain a decreased amount of water relative to nipple aspirate fluid as obtained from a patient. Such a concentration of a nipple aspirate fluid sample may be accomplished by any of a number of known means, including, for example, centrifugation and spin filtering, ultrafiltration, chromatography, ammonium sulfate precipitation, TCA/DOC, and gel electrophoresis.

Capture Assay

The assay described herein uses a capture agent to select out carbohydrates, proteins, or lipids displaying carbohydrate biomarkers from the nipple aspirate fluid sample. Carbohydrate biomarkers of interest include TF and Tn. The assays can be conducted using any procedure selected from the variety of standard assay protocols generally known in the art. As it is generally understood, the assay is constructed so as to rely on the interaction of the capture agent(s), TF and/or Tn in the sample, and labeled binding agent(s). In one embodiment, the assay utilizes some means to detect the complex formed by the capture agent(s) and the labeled binding agent(s), which allows indirect determination of the amount of TF- and/or Tn-displaying molecules within the nipple aspirate fluid sample. In another embodiment, the assay utilizes some means to detect the complex formed by the capture agent(s), the TF and/or Tn carbohydrate biomarker from the nipple aspirate fluid sample, and the labeled binding agent(s), which allows direct determination of the amount of TF- and/or Tn-displaying molecules within the nipple aspirate fluid sample. The specific design of the assay protocol is open to a wide variety of choice, and several clinical assay devices and protocols are available in the art. The capture assay described herein allows analysis of protein components expressing carbohydrate biomarker as well as non-protein components, such as carbohydrates and lipids.

The capture assay with indirect detection involves immobilizing a capture agent on a carrier (e.g., solid support or substrate), contacting the coated carrier with the nipple aspirate fluid sample, reacting the remainder of binding sites with a labeled binding agent specific for the capture agent, and detecting the label (see e.g. Example 2). The more carbohydrate biomarker of interest in the nipple aspirate fluid sample, the less tagged binding agent can attach to a given amount of capture agent on the solid support (the labeled binding agent is usually supplied in saturation compared to the amount of capture agent).

Generally, the capture assay with direct detection involves immobilizing a capture agent on a carrier, contacting the coated carrier with the nipple aspirate fluid sample, adding a labeled binding agent specific for the TF or Tn carbohydrate biomarker, and detecting the label (see e.g. Example 3). For direct detection assays, the more carbohydrate biomarker of interest in the nipple aspirate fluid sample, the more tagged binding agent can attach to the carbohydrate biomarker which is in turn bound to the capture agent on the carrier (the labeled binding agent is usually supplied in saturation).

In both indirect and direct detection methodologies, the reaction can be quantitized by comparing against a standard curve derived from a known amount(s) of non-tagged TF- and/or Tn-displaying molecules.

Capture Agent and Carrier

The capture agent of the above described assay is immobilized on a carrier and then exposed to the nipple aspirate fluid sample, from which the capture agent binds TF or Tn if present.

A TF or Tn capture agent coating a solid phase material will generally bind a sufficient quantity of TF or Tn antigen, respectively, within a relatively short period of time (approximately two to five minutes), and retain the captured TF or Tn antigen during subsequent washing and detection of labeled binding agent. The density of the capture agent on the carrier can be, for example, from about 200 ng cm⁻² to about 650 ng cm⁻². The amount of capture agent immobilized on the carrier should be in excess of the expected amount of TF or Tn in the sample. Generally, TF and Tn concentrations in undiluted cancerous nipple aspirate fluid samples range from about 15 ng/μl to about 2,500 ng/μl. Calculating the amount of capture agent to be immobilized on the carrier as a function of the expected concentration of carbohydrate antigen in the nipple aspirate fluid and the volume of sample delivered is well within the skill in the art.

Capture agents include immunoglobulin peptides, lectins, bacteriophages, or other polypeptides that bind specifically to TF and/or Tn antigen. Examples of TF-specific lectin capture agents include Amaranthus caudatus lectin; Artocarpus integrifolia Jacalin lectin; Arachis hypogea peanut lectin; and Bauhinia purpurea agglutinin. Examples of Tn-specific lectin capture agents include isolectin B4 (VVLB4) lectin and SSL lectin. Bacteriophage displaying TF-binding amino acid peptide (p-30) is another example of a capture agent (see Peletskaya (1997) J. Mol. Biol. 270, 374). Similarly, the p-30 peptide can be employed as a polypeptide capture agent. Immunoglobulin peptide capture agents include, for example, polyclonal antibodies, monoclonal antibodies, and antibody fragments such as proteolytically cleaved antibody fragments and single chain Fv antibody fragments, as further discussed below. It should be understood that the capture agents disclosed in the Examples do not limit the extent and variety of antibodies that can be used for practicing the methods described herein.

The carrier is a suitable substrate onto which the capture agent will attach, usually by electrostatic forces. The carrier can be, for example, plastic or glass material in the form of a tray, bead, or tube, or the carrier can be a suitable membrane of nylon or nitrocellulose. Preferably, the carrier is a plastic microtiter well. Immobilization onto the carrier can occur, for example, by incubating the capture agent in the microtiter well for about 4 hours at about 37° C.

After immobilization, excess capture agent is removed, and the carrier is usually blocked with albumin. For example, the carrier can be blocked with 2% BSA in 10 mM Tris-HCl buffer for six to twelve hours at 4° C. After blocking, the carrier can be washed with a suitable buffer, preferably containing a surfactant.

The nipple aspirate fluid sample solution, or dilutions thereof, is then applied to the capture agent-coated carrier under conditions in which the capture agent binds molecules that display the carbohydrate biomarker of interest. Nipple aspirate fluid can be applied, for example, at the concentration collected from the patient, or serially diluted by, for example, 1/10, 1/50, 1/100, 1/500, or 1/1000. The volume of nipple aspirate fluid supplied should be such that the amount of immobilized capture agent on the carrier is in excess to the expected amount of TF or Tn in the sample, as described above. Suitable conditions are, for example, incubation of 100 μl of diluted nipple aspirate sample for about four hours at room temperature. After allowing sufficient time for binding of carbohydrate biomarker(s) to the capture agent(s), the nipple aspirate fluid sample is then washed away.

Labeled Binding Agent

After forming the biomarker-capture agent complex, the carrier is combined with a labeled binding agent. The target of the labeled binding agent will depend upon whether indirect or direct detection means are employed.

In indirect detection assays, the resulting biomarker-capture agent complex is further reacted with a binding agent that has affinity for the capture agent, where the binding agent is attached to an easily assayable tag. The binding agent preferably binds with high affinity to immobilized capture agent not bound by carbohydrate biomarkers of interest from the nipple aspirate fluid sample but does not bind to immobilized capture agent already bound by carbohydrate biomarkers of interest from the nipple aspirate fluid sample. An example of a Tn-displaying molecule that can be used as a tagged binding agent is asialo-ovine submaxillary mucin (A-OSM) (Freire et al. (2003) Oncology Rep. 10, 1577). Examples of TF-displaying molecules that can be used as binding agents include: Asialofetuin (Sigma Chemical Co., St. Louis, Mo.); Asiaolimucin (Sigma Chemical Co., St. Louis, Mo.); Asialoglycophorin (Sigma Chemical Co., St. Louis, Mo.); and Gal beta 1,3GalNAc-alpha-O-benzyl (Sata et al. (1990) J Histochem Cytochem. 38, 763).

In direct detection assays, the resulting biomarker-capture agent complex is further reacted with a binding agent that has affinity for the carbohydrate biomarker, where the binding agent is attached to an easily assayable tag. Direct detection binding agents can include antibodies and lectins that display binding specificity for TF or Tn.

The assayable tag may be detectable directly or may bind to a reporter for which it has specificity. The assayable tag attached to the binding agent can be, for example, an enzyme, a coenzyme, an enzyme substrate, an enzyme co-factor, an enzyme inhibitor, a radionuclide, a chromogen, a fluorescer, a chemoluminescer, a free radical, or a dye. Alternatively, detection can be mediated by reporter reagents such as fluorescent avidins, streptavidins or other biotin-binding proteins or enzyme-conjugated streptavidins plus a fluorogenic, chromogenic, or chemiluminescent substrate.

Preferably, the tag is biotin, which is then recognized by avidin or streptavidin conjugated to a reporter, such as the enzyme horseradish peroxidase. For example, the tagged binding agent can be biotinylated ASF or biotinylated isolectin B4 from Vicia villosa lectin (VVLB4). Biotin is typically conjugated to proteins via primary amines (i.e., lysines). Usually, between three and six biotin molecules are conjugated to each binding agent molecule. The avidin homolog streptavidin, which is secreted by Streptomyces avidinii, is preferred as a linking agent because of its particularly high affinity for biotin.

A number of fluorescent compounds such as fluorescein isothiocyanate, europium, lucifer yellow, rhodamine β isothiocyanate (Wood (1991) In: Principles and Practice of Immunoassay, Stockton Press, New York, pp. 365-392) can be used to label binding agents. In conjunction with the known techniques for separation of antibody-antigen complexes, these fluorophores can be used to quantify TF or Tn in nipple aspirate fluid samples. The same applies to chemiluminescent immunoassay in which case either anti-TF or anti-Tn antibody can be labeled with isoluminol or acridinium esters (Krodel (1991) In: Bioluminescence and Chemiluminescence: Current Status. John Wiley and Sons Inc. New York, pp 107-110; Weeks (1983) Clin. Chem. 29:1480-1483). Radioimmunoassay (Kashyap, M. L. et al., J. Clin. Invest. 60:171-180 (1977)) is another technique in which anti-TF or anti-Tn antibodies can be used after coupling with a radioactive isotope such as ¹²⁵I. Some of these immunoassays can be easily automated by the use of appropriate instruments such as the IMX™ (Abbott, Irving, Tex.) for a fluorescent immunoassay and Ciba Corning ACS 180™ (Ciba Corning, Medfield, Mass.) for a chemiluminescent immunoassay. Kits for detection of tagged binding agents in ELISA procedures are commercially available.

Detection

Detection follows washing away unbound labeled binding agent. In the indirect detection approach, the tag in the complex formed from the capture agent and tagged binding agent is detected, thereby indirectly indicating the amount of TF or Tn present. Thus, for indirect detection methods, when the carbohydrate biomarker of interest is present in the nipple aspirate fluid sample, low signal will be detected from the label as there will have been fewer available sites for the labeled binding agent to bind.

Alternatively, in the direct detection approach, the tag in the complex formed from the capture agent, carbohydrate biomarker of interest, and the tagged binding agent is detected, thereby directly indicating the amount of TF or Tn present. Thus, for direct detection methods, when the carbohydrate biomarker of interest is present in the nipple aspirate fluid sample, high signal will be detected from the label as there will have been more sites for the labeled binding agent to bind.

Detection methodology will depend upon the identity of the assayable tag on the binding agent, as commonly understood in the art. Kits for detection of tagged binding agents in the capture immunoassay described above are commercially available. Detection procedures include Western blots, enzyme-linked immunosorbent assays, radioimmunoassays, competition immunoassays, dual antibody sandwich assays, immunohistochemical staining assays, agglutination assays, and fluorescent immunoassays.

Preferably, a streptavidin/peroxidase complex is used to assay the amount of biotin tag. The activity of the peroxidase enzyme linked to the streptavidin can then be detected through the addition of a peroxidase substrate. An example of a peroxidase substrate is 2,2′-Azino-bis(3-ethyl benzthiazoline-6-sulfonic acid) (ABTS).

Solutions with known amounts of carbohydrate biomarkers can be used in the generation of standard curves. An example of a Tn-displaying molecule that can be used as a standard for determining the concentration of Tn in nipple aspirate fluid samples is A-OSM (Freire et al. (2003) Oncology Rep. 10:1577). Examples of TF-displaying molecules that can be used as standards for determining the concentration of TF in nipple aspirate fluid samples include: Asialofetuin (Sigma Chemical Co., St. Louis, Mo.); Asiaolimucin (Sigma Chemical Co., St. Louis, Mo.); Asialoglycophorin (Sigma Chemical Co., St. Louis, Mo.) and Gal beta 1,3GalNAc-alpha-O-benzyl (Sata et al. (1990) J Histochem Cytochem. 38, 763).

Lectins

Lectins can be used as capture agents and/or binding agents. Generally, antigen-specific lectins can be used as either capture agents or binding agents in direct detection methods, and as capture agents in indirect detection methods. A lectin is a carbohydrate-binding protein of non-immune origin that agglutinates cells or precipitates glycoconjugates. Lectins can be isolated from many types of organisms including plants, viruses, microorganisms, and animals. Lectins are generally multimeric, consisting of non-covalently associated subunits. A lectin may contain two or more of the same subunit, such as Concanavalin A, or different subunits, such as Phaseolus vulgaris agglutinin. It is this multimeric structure which gives lectins their ability to agglutinate cells or form precipitates with glycoconjugates in a manner similar to antigen-antibody interactions. A wide array of lectins is commercially available. Examples of Tn-specific lectins include: Isolectin VVB4 (anti-Tn-reactive isolectin B4 from Vicia villosa) (Vector Labs, Burlinagame, Calif.); SSL Lectin (Medeiros et al. (2000) Eur. J. Biochem. 267: 1434); and Macrophage c-type lectin (lida et al. (1999) J. Biol. Chem. 274: 10697). Examples of TF-specific lectins include: Amaranthus caudatus lectin (ACL); Artocarpus integrifolia (Jacalin); Arachis hypogea (peanut lectin, PNL); and Bauhinia purpurea agglutinin (BPA) (Vector Labs, Burlingame, Calif.). For use as a binding agent, the lectins listed above are linked to an easily assayable tag (see e.g. Example 3).

Immunoglobulin Peptides

Immunoglobulin peptides can be used as capture agents and/or binding agents. Immunoglobulin peptides include, for example, polyclonal antibodies, monoclonal antibodies, and antibody fragments. Immunoglobulin peptides used as capture agents are immobilized on a substrate surface, as described above. Immunoglobulin peptides used as binding agents have easily assayed labels or tags affixed, as described above. The following describes generation of Immunoglobulin peptides, specifically TF and Tn antibodies, via methods that can be used by those skilled in the art to make other suitable Immunoglobulin peptides having similar affinity and specificity which are functionally equivalent to those used in the Examples.

Polyclonal antibodies may be readily generated by one of ordinary skill in the art from a variety of warm-blooded animals such as horses, cows, various fowl, rabbits, mice, or rats. Briefly, TF or Tn antigen is utilized to immunize the animal through intraperitoneal, intramuscular, intraocular, or subcutaneous injections, with an adjuvant such as Freund's complete or incomplete adjuvant. Following several booster immunizations, samples of serum are collected and tested for reactivity to TF or Tn. Particularly preferred polyclonal antisera will give a signal on one of these assays that is at least three times greater than background. Once the titer of the animal has reached a plateau in terms of its reactivity to TF or Tn, larger quantities of antisera may be readily obtained either by weekly bleedings, or by exsanguinating the animal.

Monoclonal antibody (MAb) technology can be used to obtain MAbs to rapidly and reliably quantitate TF and Tn antigens in nipple aspirate fluid samples. Briefly, hybridomas are produced using spleen cells from mice immunized with TF or Tn antigens. The spleen cells of each immunized mouse are fused with mouse myeloma Sp 2/0 cells, for example, using the polyethylene glycol fusion method of Galfre, G. and Milstein, C. (1981) Methods Enzymol. 73:3-46. Growth of hybridomas, selection in HAT medium, cloning, and screening of clones against antigens are carried out using standard methodology (Galfre, G. and Milstein, C. (1981) Methods Enzymol. 73:3-46).

HAT-selected clones are injected into mice to produce large quantities of MAb in ascites as described by Galfre, G. and Milstein, C. (1981) Methods Enzymol. 73:3-46), which can be purified using protein A column chromatography (BioRad, Hercules, Calif.). MAbs are selected on the basis of their (a) specificity for TF or Tn, (b) high binding affinity, (c) isotype, and (d) stability.

MAbs can be screened or tested for specificity using any of a variety of standard techniques, including Western Blotting (Koren, E. et al. (1986) Biochim. Biophys. Acta 876:91-100) and enzyme-linked immunosorbent assay (ELISA) (Koren, E. et al. (1986) Biochim. Biophys. Acta 876:91-100).

Examples of Tn-specific MAbs include: Tn MAb B1.1 (IgM, V 1053) (Biomeda, Foster City, Calif.); Tn MAb 12A8-C7-F5 (Cao et al. (1996) Histochem cell Biol. 106: 197); TEC-02 (Draber (1987) Cell Differ. 21: 119); MAb CD175 (IgM) (DBS, Pleasanton, Calif.); Anti Tn Ab-MLS 128 (Nakada et al. (1993) PNAS 90: 2495); Anti Tn MAb 83D4 (Freire et al. (2003) Oncology Rep. 10: 1577); MAb B72.3 (Takada et al. (1993) Cancer Res. 53: 354); and MAb B 230.9 (Reddish at al. (1997) Glycoconjugate J. 14: 549).

Examples of TF-specific MAbs include: MAbA78-G/A7 (IgM) (Neomarkers, Fremont, Calif.); MAb 49H.8 (Murine) (Longnecker et al. (1990) Cancer Res. 50: 4801); MAb A68-B/A11 (IgM) (Kamiya Biomedical Comp. Seattle, Wash.); MAb B386 (IgM) (Biomeda, Foster City, Calif.); MAb HB-T1 (DAKO Corp. Hamburg, Germany); MAb BM22 (Murine) (DAKO Corp. Hamburg, Germany); MAB HH8 (DAKO Corp. Hamburg, Germany); MAb RS1-114 (DAKO Corp. Hamburg, Germany); MAb TF1 (IgM, k) (BioInvent, Lund, Sweden); MAb TF2 (IgA, k) (BioInvent, Lund, Sweden); MAb TF5 (IgM, lambda) (BioInvent, Lund, Sweden); MAb 5A8 (IgM, Murine) (BioInvent, Lund, Sweden); MAb 8D8 (IgM, Murine) (BioInvent, Lund, Sweden); Mab CC49 (murine) (Schlom et al. (1992) Cancer Research 52: 1067-1072); Mab B72.3 (Murine) (Abcam Inc, Cambridge, Mass.); and Humanized CC49 (Kashmiri et al. (1995) Hydriboma 14: 461-473).

It may be desirable to produce and use functional fragments of a MAb for a particular application. The well-known basic structure of a typical IgG molecule is a symmetrical tetrameric Y-shaped molecule of approximately 150,000 to 200,000 daltons consisting of two identical light polypeptide chains (containing about 220 amino acids) and two identical heavy polypeptide chains (containing about 440 amino acids). Heavy chains are linked to one another through at least one disulfide bond. Each light chain is linked to a contiguous heavy chain by a disulfide linkage. An antigen-binding site or domain is located in each arm of the Y-shaped antibody molecule and is formed between the amino terminal regions of each pair of disulfide linked light and heavy chains. These amino terminal regions of the light and heavy chains consist of approximately their first 110 amino terminal amino acids and are known as the variable regions of the light and heavy chains. In addition, within the variable regions of the light and heavy chains there are hypervariable regions which contain stretches of amino acid sequences, known as complementarity determining regions (CDRs). CDRs are responsible for the antibody's specificity for one particular site on an antigen molecule called an epitope. Thus, the typical IgG molecule is divalent in that it can bind two antigen molecules because each antigen-binding site is able to bind the specific epitope of each antigen molecule. The carboxy terminal regions of light and heavy chains are similar or identical to those of other antibody molecules and are called constant regions. The amino acid sequence of the constant region of the heavy chains of a particular antibody defines what class of antibody it is, for example, IgG, IgD, IgE, IgA or IgM. Some classes of antibodies contain two or more identical antibodies associated with each other in multivalent antigen-binding arrangements.

Proteolytic cleavage of a typical IgG molecule with papain is known to produce two separate antigen binding fragments called Fab fragments which contain an intact light chain linked to an amino terminal portion of the contiguous heavy chain via by disulfide linkage. The remaining portion of the papain-digested immunoglobin molecule is known as the Fc fragment and consists of the carboxy terminal portions of the antibody left intact and linked together via disulfide bonds. If an antibody is digested with pepsin, a fragment known as an F(ab′)₂ fragment is produced which lacks the Fc region but contains both antigen-binding domains held together by disulfide bonds between contiguous light and heavy chains (as Fab fragments) and also disulfide linkages between the remaining portions of the contiguous heavy chains (Handbook of Experimental Immunology. Vol 1: Immunochemistry, Weir, D. M., Editor, Blackwell Scientific Publications, Oxford (1986)).

Fab and F(ab′)₂ fragments of MAbs that bind TF or Tn can be used in place of whole MAbs in methods for detecting or quantifying TF or Tn antigen in nipple aspirate fluid samples. Because Fab and F(ab′)₂ fragments are smaller than intact antibody molecules, more antigen-binding domains can be immobilized per unit area of a solid support than when whole antibody molecules are used. As explained below, rapid, easy, and reliable assay systems can be made in which antibodies or antibody fragment that specifically bind TF or Tn are immobilized on solid phase materials.

Recombinant DNA methods have been developed which permit the production and selection of recombinant immunoglobulin peptides which are single chain antigen-binding polypeptides known as single chain Fv fragments (ScFvs or ScFv antibodies). ScFvs bind a specific epitope of interest and can be produced using any of a variety of recombinant bacterial phage-based methods, for example as described in Lowman et al. (1991) Biochemistry, 30: 10832-10838; Clackson et al. (1991) Nature 352: 624-628; and Cwirla et al. (1990) Proc. Natl. Acad. Sci. USA 87: 6378-6382. These methods are usually based on producing genetically altered filamentous phage, such as recombinant M13 or fd phages, which display on the surface of the phage particle a recombinant fusion protein containing the antigen-binding ScFv antibody as the amino terminal region of the fusion protein and the minor phage coat protein g3p as the carboxy terminal region of the fusion protein. Such recombinant phages can be readily grown and isolated using well-known phage methods. Furthermore, the intact phage particles can usually be screened directly for the presence (display) of an antigen-binding ScFv on their surface without the necessity of isolating the ScFv away from the phage particle.

To produce an ScFv, standard reverse transcriptase protocols are used to first produce cDNA from mRNA isolated from a hybridoma that produces an MAb for TF or Tn antigen. The cDNA molecules encoding the variable regions of the heavy and light chains of the MAb can then be amplified by standard polymerase chain reaction (PCR) methodology using a set of primers for mouse immunoglobulin heavy and light variable regions (Clackson (1991) Nature 352: 624-628). The amplified cDNAs encoding MAb heavy and light chain variable regions are then linked together with a linker oligonucleotide in order to generate a recombinant ScFv DNA molecule. The ScFv DNA is ligated into a filamentous phage plasmid designed to fuse the amplified cDNA sequences into the 5′ region of the phage gene encoding the minor coat protein called g3p. Escherichia coli bacterial cells are than transformed with the recombinant phage plasmids, and filamentous phage grown and harvested. The desired recombinant phages display antigen-binding domains fused to the amino terminal region of the minor coat protein. Such “display phages” can then be passed over immobilized antigen, for example, using the method known as “panning”, see Parmley and Smith (1989) Adv. Exp. Med. Biol. 251: 215-218; Cwirla et al. (1990) Proc. Natl. Acad. Sci. USA 87: 6378-6382, to adsorb those phage particles containing ScFv antibody proteins that are capable of binding antigen. The antigen-binding phage particles can then be amplified by standard phage infection methods, and the amplified recombinant phage population again selected for antigen-binding ability. Such successive rounds of selection for antigen-binding ability, followed by amplification, select for enhanced antigen-binding ability in the ScFvs displayed on recombinant phages. Selection for increased antigen-binding ability may be made by adjusting the conditions under which binding takes place to require a tighter binding activity. Another method to select for enhanced antigen-binding activity is to alter nucleotide sequences within the cDNA encoding the binding domain of the ScFv and subject recombinant phage populations to successive rounds of selection for antigen-binding activity and amplification (see Lowman et al. (1991) Biochemistry 30: 10832-10838; and Cwirla et al. (1990) Proc. Natl. Acad. Sci. USA 87: 6378-6382).

Once a ScFv is selected, the recombinant TF or Tn antibody can be produced in a free form using an appropriate vector in conjunction with E. coli strain HB2151. These bacteria actually secrete ScFv in a soluble form, free of phage components (Hoogenboom et al. (1991) Nucl. Acids Res. 19: 4133-4137). The purification of soluble ScFv from the HB2151 bacteria culture medium can be accomplished by affinity chromatography using antigen molecules immobilized on a solid support such as AFFIGEL™ (BioRad, Hercules, Calif.).

Other developments in the recombinant antibody technology demonstrate possibilities for further improvements such as increased avidity of binding by polymerization of ScFvs into dimers and tetramers (see Holliger et al. (1993) Proc. Natl. Acad. Sci. USA 90: 6444-6448).

Because ScFvs are even smaller molecules than Fab or F(ab′)₂ fragments, they can be used to attain even higher densities of antigen binding sites per unit of surface area when immobilized on a solid support material than possible using whole antibodies, F(ab′)₂, or Fab fragments. Furthermore, recombinant antibody technology offers a more stable genetic source of antibodies, as compared with hybridomas. Recombinant antibodies can also be produced more quickly and economically using standard bacterial phage production methods.

Kits

The capture agent(s), labeled binding agent(s), revealing reagents, and/or standards for the conduct of the various capture immunoassays described herein may conveniently be supplied as kits which include the necessary components and instructions for the assay. Screening/diagnositic kits typically comprise one or more reagents that specifically bind to the target that is to be screened (e.g. ligands that specifically bind to TF- or Tn-antigens). The reagents can, optionally, be provided with an attached label and/or affixed to a substrate (e.g. as a component of a protein array), and/or can be provided in solution. The kits can comprise nucleic acid constructs (e.g. vectors) that encode one or more such ligands to facilitate recombinant expression of such. The kits can optionally include one or more buffers, detectable labels or labeled binding agents, or other reagents as may be useful in a particular assay.

In addition, the kits optionally include labeling and/or instructional materials providing directions (i.e., protocols) for the practice of the methods described herein. Preferred instructional materials describe the detection of TF- and Tn-antigens in nipple aspirate fluid samples for the diagnosis, staging, and/or prognosis of breast cancer. While the instructional materials typically comprise written or printed materials, they are not limited to such. Any medium capable of storing such instructions and communicating them to an end user is contemplated. Such media include, but are not limited to electronic storage media (e.g., magnetic discs, tapes, cartridges, chips), optical media (e.g., CD ROM), and the like. Such media may include addresses to internet sites that provide such instructional materials.

A preferred kit includes a microtiter plate coated with a TF- or Tn-specific antibody, standard solutions for preparation of standard curve, a control for quality testing of the analytical run, TF and/or Tn antigens conjugated to biotin, streptavidin-peroxidase enzyme, a substrate solution, a stopping solution, a washing buffer, and an instruction manual.

Having described the invention in detail, it will be apparent that modifications and variations are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

Nipple aspirate fluid (NAF) was collected noninvasively using a modified breast pump. The nipple was cleansed with alcohol. A warm, moist cloth was placed on the breast after the alcohol evaporated. The cloth was removed after 2 minutes. While the breast was massaged, a syringe connected to the breast pump collected NAF. Aspiration was repeated on the opposite breast, if present. Fluid in the form of droplets (1-200 μl) was collected in capillary tubes, and the samples were immediately snap frozen at −80° C.

### Example 2

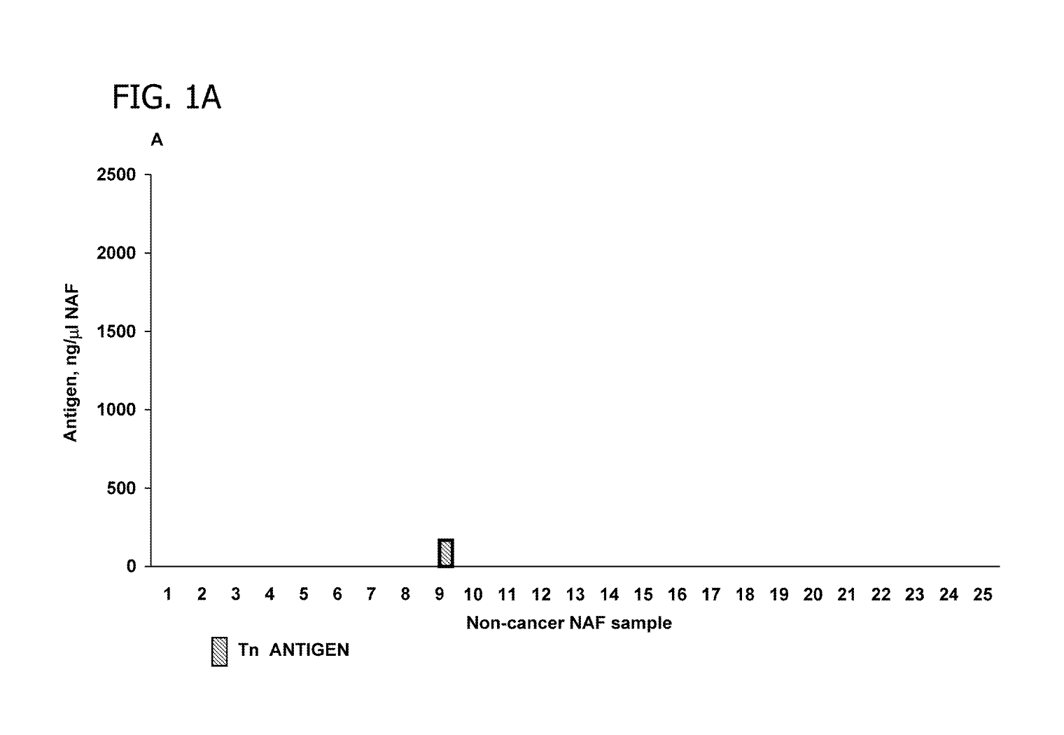

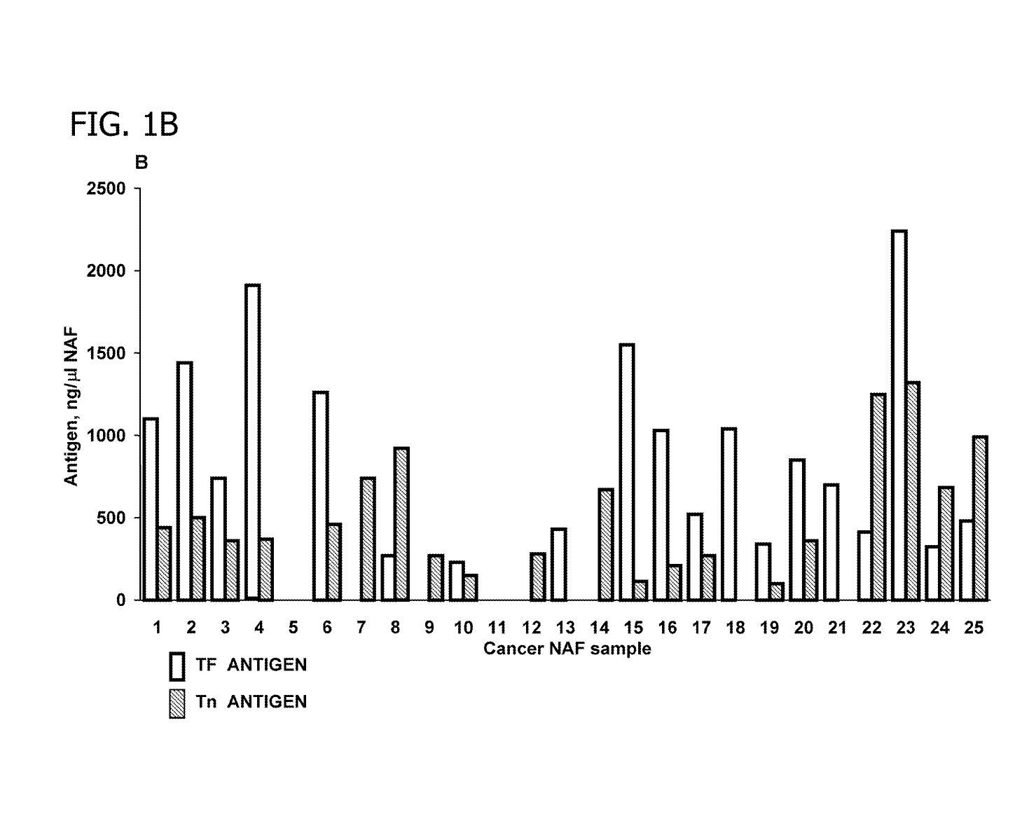

TF and TN antigen in NAF samples were quantitated by an antigen capture immunoassay employing indirect detection as follows. For TF antigen quantitation, microtiter wells (Immunomaxi, Switzerland) were coated with 50 μl of anti-TF antibody A78-G/A7 (1 μg/ml in 0.1 M carbonate buffer, pH 9.6) by incubating the plates at 37° C., for 4 hours. After removing the excess antibody, the wells were blocked with 2% BSA in 10 mM Tris-HCl buffer overnight at 4° C. in a humid chamber. After overnight incubation, the plate wells were washed three times with Tris buffered saline containing 0.1% Tween-20 (TTBS), using an automatic plate washer (Elx405, BIO-TEK). To the washed wells, 100 μl of appropriately diluted control and cancer NAF samples were added and incubated for 4 hours at room temperature (RT). After washing the wells, 50 μl of biotinylated ASF (2.0 mg/ml) was added to the wells and incubated for 1 hour at RT. Unbound ASF was then washed and 100 μl of 1/2000 streptavidin/peroxidase complex (Sigma) in TTBS buffer was added and incubated at RT for 45-60 minutes. Peroxidase activity was demonstrated by incubation in ABTS (2,2-Azinbis(3-ethylbenzthiazoline-6-sulfonic acid)) liquid substrate system (Sigma). The reaction was allowed to proceed for 30 minutes during which time the absorbance was read at 405 nm with an ELISA plate reader (Bio-Tek, Vermont). Sample concentrations of TF antigen were determined by interpolation against a standard curve performed with a series of known concentrations of biotinylated asialofetuin (ASF) (see e.g. FIGS. 1A and 1B). All experimental samples were performed and analyzed in duplicate. Values <15 ng/μL were considered as 0 (not detectable). Statistical significance between the values obtained for non-cancer and cancer NAF values was determined by student's t test.

Tn antigen in NAF samples were quantitated in an antigen capture immunoassay, using an anti-Tn MAb to capture epitope positive molecules, asialo-ovine submaxillary mucin (A-OSM), and biotinylated isolectin B4 from Vicia villosa lectin (VVLB4) as the detection molecule. Briefly, the microtiter wells were coated with 50 μl of Tn MAb V-1053 (5 μg/ml in 0.1 M carbonate buffer, pH 9.6) by overnight incubation at RT. The wells were washed with 0.1% Tween 20 in TBS, and incubated with 1% gelatin in TBS at 37° C. for 1 hour. After three washes, wells were incubated with 100 μl of NAF samples serially diluted in TBS, overnight at 4° C. After washing, the wells were incubated with 50 μl of 1/250 A-OSM for 3 hours at RT. A-OSM was the Tn-positive ligand used to develop the anti-Tn antibody V-1053. Unbound material was then washed off and the wells were incubated with biotinylated VVLB4 (5 μg/ml) in 0.5% gelatin, 0.1% Tween 20 in TBS, at 37° C. for 1 hour. After washing the wells, 100 μl of 1/2000 avidin/peroxidase complex in TBS was added and incubated for 1 hour at 37° C. Peroxidase activity was estimated as described for TF antigen. Sample concentrations of Tn antigen were determined by interpolation against a standard curve performed with different concentrations of asialo-ovine submaxillary mucin (see e.g. FIGS. 1A and 1B). Values <15 ng/μL were considered as 0 (not detectable). All experimental samples were analyzed in duplicate. Statistical significance between the values obtained for non-cancer and cancer NAF values was determined by student's t test.

The results showed that the presence of TF and Tn correlated with disease presence. Background levels of TF and Tn antigen in healthy volunteers were very low except for one individual, and it was later determined that this individual in fact had cancer in the other, non-sampled, breast. Statistical analyses demonstrated that the differences detected between cancer patients and healthy volunteers were significant, i.e., TF and Tn antigens are elevated in cancer patient NAF (TF, P=0.0007; Tn, P=0.0002) (see e.g. FIG. 1B) and very low or non-detectable levels in healthy patients (see FIG. 1A). NAF samples were obtained from patients that had stage 0-4 disease with ductal and lobular location with and without lymph node metastases. There did not appear to be a correlation between disease stage or location and the expression level of either TF or Tn.

### Example 3

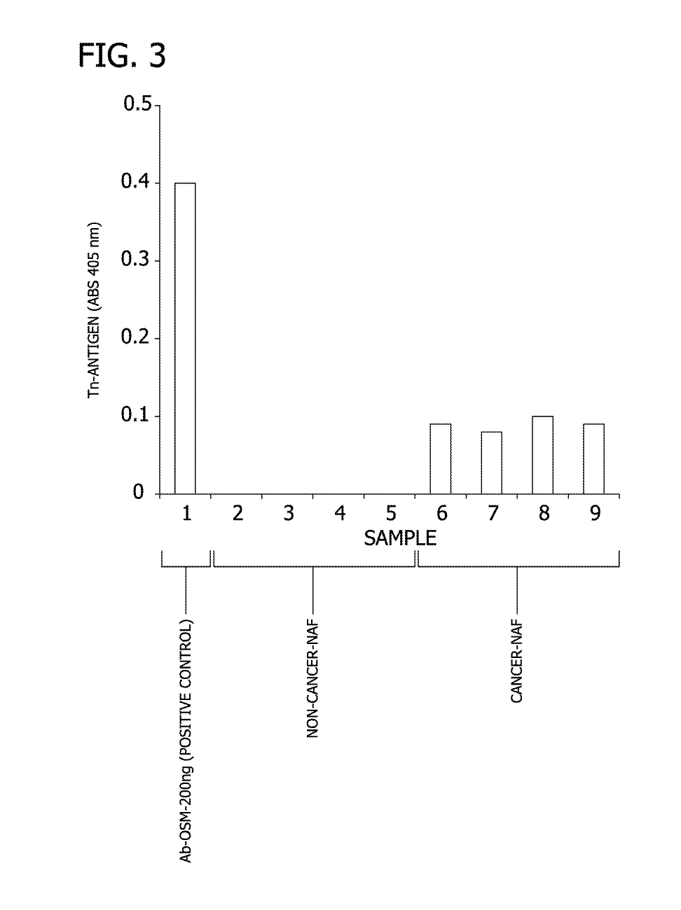

Tn antigen in NAF samples were quantitated by an antigen capture immunoassay employing direct detection as follows. The well of a microtiter plate was coated with anti-Tn antibody, blocked and rinsed as described above. NAF sample from cancerous and non-cancerous patients, diluted one to fifty, was added (100 μl) to the wells as described above. The plates were rinsed and biotinylated isolectin VVL-4B was added and allowed to incubate for approximately one hour at 25° C. The plates were then rinsed and streptavidin conjugated alkaline phosphatase was added and allowed to incubate for approximately one hour at 25° C. Chromagenic alkaline phosphatase substrate was added and the developed color was assayed by reading the optical density of the sample. Results showed that in NAF samples of non-cancerous patients, no significant Tn was detected, while in NAF samples from cancerous patients, Tn was detected with biotinylated lectin (see e.g. FIG. 3).

What is claimed is:

1. A method for detection of biomarkers comprising assaying nipple aspirate fluid (NAF) derived from a subject for the presence of a carbohydrate biomarker occurring on protein, lipid or carbohydrate molecules dissolved in the NAF, said carbohydrate biomarker being selected from the group consisting of TF, Tn, and both TF and Tn.

2. The method of claim 1 wherein assaying NAF comprises the steps of:
combining a NAF sample with a capture agent bound to a carrier, wherein the capture agent binds specifically to the carbohydrate biomarker when present in the NAF sample; and
detecting the carbohydrate biomarker bound to the capture agent.

3. The method of claim 2 wherein the carbohydrate biomarker is detected indirectly by:
placing a labeled binding agent in contact with the carrier and the carrier-bound capture agent, wherein the labeled binding agent binds specifically to carrier-bound capture agent that is not already bound to the carbohydrate biomarker from the NAF sample but the labeled binding agent does not bind to carrier-bound capture agent that is already bound to carbohydrate biomarker from the NAF sample; and
detecting the labeled binding agent that is bound to the capture agent.

4. The method of claim 3 wherein:
the NAF sample is combined with a first and a second capture agent bound to a carrier such that the first capture agent binds specifically to TF carbohydrate biomarker when present in the NAF sample and the second capture agent binds specifically to Tn carbohydrate biomarker when present in the NAF sample;
a first and a second labeled binding agent is contacted with the first and the second carrier-bound capture agent such that (i) the first labeled binding agent binds specifically to a first capture agent that is not already bound to TF carbohydrate biomarker from the NAF sample but the first labeled binding agent does not bind to a first capture agent that is already bound to TF carbohydrate biomarker from the NAF sample and (ii) the second labeled binding agent binds specifically to a second capture agent that is not already bound to Tn carbohydrate biomarker from the NAF sample but the second labeled binding agent does not bind to a second capture agent that is already bound to Tn carbohydrate biomarker from the NAF sample; and
the first and the second labeled binding agents that are bound to the TF- and Tn-capture agents, respectively, are detected.

5. The method of claim 2 wherein the presence of the carbohydrate biomarker is detected directly by:
placing a labeled binding agent in contact with the carrier and the carrier-bound capture agent, wherein the labeled binding agent binds specifically to the carbohydrate biomarker from the NAF sample that is in turn bound to the capture agent; and
detecting the labeled binding agent that is bound to the carbohydrate biomarker.

6. The method of claim 5 wherein:
the NAF sample is combined with a first and a second capture agent bound to a carrier such that the first capture agent binds specifically to TF carbohydrate biomarker when present in the NAF sample and the second capture agent binds specifically to Tn carbohydrate biomarker when present in the NAF sample;
a first and a second labeled binding agent is contacted with the first and the second carrier-bound capture agent such that (i) the first labeled binding agent binds specifically to a TF carbohydrate biomarker from the NAF sample that is in turn bound to the first capture agent; and (ii) the second labeled binding agent binds specifically to a Tn carbohydrate biomarker from the NAF sample that is in turn bound to the second capture agent; and
the first and the second labeled binding agents that are bound to TF carbohydrate biomarker and Tn carbohydrate biomarker, respectively, are detected.

7. The method of claim 2 wherein the capture agent is selected from the group consisting of an antibody, an antigen binding fragment, and a lectin.

8. The method of claim 3 wherein the labeled binding agent for indirect detection is a labeled TF-specific antigen or a labeled Tn-specific antigen.

9. The method of claim 5 wherein the labeled binding agent for direct detection is a labeled TF-specific lectin or a labeled Tn-specific lectin.

10. The method of claim 5 wherein the step of detecting the carbohydrate biomarker comprises quantifying the amount of labeled binding agent.

11. A method for detection of biomarkers comprising assaying nipple aspirate fluid (NAF) or a dilution or concentrate thereof derived from a subject for the presence of a secreted carbohydrate biomarker, said carbohydrate marker being selected from the group consisting of TF, Tn, and both TF and Tn, said method comprising the steps of:
combining a NAF or NAF dilution or concentrate sample with a capture agent bound to a carrier, wherein the capture agent binds specifically to the secreted carbohydrate biomarker when present in the NAF or NAF dilution or concentrate sample; and
detecting the carbohydrate biomarker bound to the capture agent.

12. The method of claim 11 wherein the carbohydrate biomarker is detected indirectly by:
placing a labeled binding agent in contact with the carrier and the carrier-bound capture agent, wherein the labeled binding agent binds specifically to carrier-bound capture agent that is not already bound to the carbohydrate biomarker from the NAF or NAF dilution or concentrate sample but the labeled binding agent does not bind to carrier-bound capture agent that is already bound to carbohydrate biomarker from the NAF or NAF dilution or concentrate sample; and
detecting the labeled binding agent that is bound to the capture agent.

13. The method of claim 12 wherein:
the NAF or NAF dilution or concentrate sample is combined with a first and a second capture agent bound to a carrier such that the first capture agent binds specifically to TF carbohydrate biomarker when present in the NAF or NAF dilution or concentrate sample and the second capture agent binds specifically to Tn carbohydrate biomarker when present in the NAF or dilution or concentrate sample;
a first and a second labeled binding agent is contacted with the first and the second carrier-bound capture agent such that (i) the first labeled binding agent binds specifically to a first capture agent that is not already bound to TF carbohydrate biomarker from the NAF or NAF dilution or concentrate sample but the first labeled binding agent does not bind to a first capture agent that is already bound to TF carbohydrate biomarker from the NAF or NAF dilution or concentrate sample and (ii) the second labeled binding agent binds specifically to a second capture agent that is not already bound to Tn carbohydrate biomarker from the NAF or NAF dilution or concentrate sample but the second labeled binding agent does not bind to a second capture agent that is already bound to Tn carbohydrate biomarker from the NAF or NAF dilution or concentrate sample; and
the first and the second labeled binding agents that are bound to the TF- and Tn-capture agents, respectively, are detected.

14. The method of claim 11 wherein the presence of the carbohydrate biomarker is detected directly by:
placing a labeled binding agent in contact with the carrier and the carrier-bound capture agent, wherein the labeled binding agent binds specifically to the carbohydrate biomarker from the NAF or NAF dilution or concentrate sample that is in turn bound to the capture agent; and
detecting the labeled binding agent that is bound to the carbohydrate biomarker.

15. The method of claim 14 wherein:
the NAF or NAF dilution or concentrate sample is combined with a first and a second capture agent bound to a carrier such that the first capture agent binds specifically to TF carbohydrate biomarker when present in the NAF or NAF dilution or concentrate sample and the second capture agent binds specifically to Tn carbohydrate biomarker when present in the NAF dilution or concentrate sample;
a first and a second labeled binding agent is contacted with the first and the second carrier-bound capture agent such that (i) the first labeled binding agent binds specifically to a TF carbohydrate biomarker from the NAF or NAF dilution or concentrate sample that is in turn bound to the first capture agent; and (ii) the second labeled binding agent binds specifically to a Tn carbohydrate biomarker from the NAF or NAF dilution or concentrate sample that is in turn bound to the second capture agent; and
the first and the second labeled binding agents that are bound to TF carbohydrate biomarker and Tn carbohydrate biomarker, respectively, are detected.

16. The method of claim 11 wherein the capture agent is selected from the group consisting of an antibody, an antigen binding fragment thereof, and a lectin.

17. The method of claim 12 wherein the labeled binding agent for indirect detection is a labeled TF-specific antigen or a labeled Tn-specific antigen.

18. The method of claim 14 wherein the labeled binding agent for direct detection is a labeled TF-specific lectin or a labeled Tn-specific lectin.

19. The method of claim 14 wherein the step of detecting the carbohydrate biomarker comprises quantifying the amount of labeled binding agent.

20. A kit for detection of a carbohydrate biomarker in nipple aspirate fluid (NAF) or NAF derivative, said kit comprising a capture agent capable of binding specifically to TF or Tn, a labeled binding agent, and instructions for use of the kit in a method according to-claim 1.

21. A method for detection of biomarkers comprising assaying nipple aspirate fluid (NAF) for a carbohydrate biomarker displayed by a secreted protein or lipid in the NAF, said carbohydrate biomarker being selected from the group consisting of TF, Tn, and both TF and Tn.

22. The method of claim 21 wherein assaying NAF comprises the steps of:
combining a NAF sample with a capture agent bound to a carrier, wherein the capture agent binds specifically to the carbohydrate biomarker when present in the NAF sample; and
detecting the carbohydrate biomarker bound to the capture agent.

23. The method of claim 22 wherein the carbohydrate biomarker is detected indirectly by:
placing a labeled binding agent in contact with the carrier and the carrier-bound capture agent, wherein the labeled binding agent binds specifically to carrier-bound capture agent that is not already bound to the carbohydrate biomarker from the NAF sample but the labeled binding agent does not bind to carrier-bound capture agent that is already bound to carbohydrate biomarker from the NAF sample; and
detecting the labeled binding agent that is bound to the capture agent.

24. The method of claim 23 wherein:
the NAF sample is combined with a first and a second capture agent bound to a carrier such that the first capture agent binds specifically to TF carbohydrate biomarker when present in the NAF sample and the second capture agent binds specifically to Tn carbohydrate biomarker when present in the NAF sample;
a first and a second labeled binding agent is contacted with the first and the second carrier-bound capture agent such that (i) the first labeled binding agent binds specifically to a first capture agent that is not already bound to TF carbohydrate biomarker from the NAF sample but the first labeled binding agent does not bind to a first capture agent that is already bound to TF carbohydrate biomarker from the NAF sample and (ii) the second labeled binding agent binds specifically to a second capture agent that is not already bound to Tn carbohydrate biomarker from the NAF sample but the second labeled binding agent does not bind to a second capture agent that is already bound to Tn carbohydrate biomarker from the NAF sample; and
the first and the second labeled binding agents that are bound to the TF- and Tn-capture agents, respectively, are detected.

25. The method of claim 22 wherein the presence of the carbohydrate biomarker is detected directly by:
placing a labeled binding agent in contact with the carrier and the carrier-bound capture agent, wherein the labeled binding agent binds specifically to the carbohydrate biomarker from the NAF sample that is in turn bound to the capture agent; and
detecting the labeled binding agent that is bound to the carbohydrate biomarker.

26. The method of claim 25 wherein:
the NAF sample is combined with a first and a second capture agent bound to a carrier such that the first capture agent binds specifically to TF carbohydrate biomarker when present in the NAF sample and the second capture agent binds specifically to Tn carbohydrate biomarker when present in the NAF sample;
a first and a second labeled binding agent is contacted with the first and the second carrier-bound capture agent such that (i) the first labeled binding agent binds specifically to a TF carbohydrate biomarker from the NAF sample that is in turn bound to the first capture agent; and (ii) the second labeled binding agent binds specifically to a Tn carbohydrate biomarker from the NAF sample that is in turn bound to the second capture agent; and
the first and the second labeled binding agents that are bound to TF carbohydrate biomarker and Tn carbohydrate biomarker, respectively, are detected.

27. The method of claim 22 wherein the capture agent is selected from the group consisting of an antibody, an antigen binding fragment thereof, and a lectin.

28. The method of claim 23 wherein the labeled binding agent for indirect detection is a labeled TF-specific antigen or a labeled Tn-specific antigen.

29. The method of claim 25 wherein the labeled binding agent for direct detection is a labeled TF-specific lectin or a labeled Tn-specific lectin.

30. The method of claim 25 wherein the step of detecting the carbohydrate biomarker comprises quantifying the amount of labeled binding agent.

31. The method of claim 11 wherein the secreted carbohydrate biomarker is displayed on a secreted protein or lipid.

32. The method of claim 12 wherein the secreted carbohydrate biomarker is displayed on a secreted protein or lipid.

33. The method of claim 13 wherein the secreted carbohydrate biomarker is displayed on a secreted protein or lipid.

34. The method of claim 14 wherein the secreted carbohydrate biomarker is displayed on a secreted protein or lipid.

35. The method of claim 15 wherein the secreted carbohydrate biomarker is displayed on a secreted protein or lipid.

36. The method of claim 16 wherein the secreted carbohydrate biomarker is displayed on a secreted protein or lipid.

37. The method of claim 17 wherein the secreted carbohydrate biomarker is displayed on a secreted protein or lipid.

38. The method of claim 18 wherein the secreted carbohydrate biomarker is displayed on a secreted protein or lipid.

39. The method of claim 19 wherein the secreted carbohydrate biomarker is displayed on a secreted protein or lipid.
